(19) United States
(12) Patent Application Publication
(10) Pub. No.: US 2011/0245704 A1
(43) Pub. Date: Oct. 6, 2011

(54) METHODS AND SYSTEMS FOR ANALYSING RESUSCITATION

(76) Inventors: Koen Monsieurs, Willebroek, (BE); Alain Kalmar, Wichelen (BE)

(21) Appl. No.: 13/139,067

(22) PCT Filed: Dec. 10, 2009
(86) PCT No.: PCT/EP2009/066851

§ 371 Date: Jun. 10, 2011

(30) Foreign Application Priority Data
Dec. 11, 2008 (GB) 0822592.2

(51) Int. Cl. A61B 5/08

(52) U.S. Cl. 600/529

(57) ABSTRACT

A system (100) for analysing resuscitation is described. The system comprises an input means (120) for obtaining a plurality of pressure values over time. It also comprises a tracheal pressure value processing means (140), and a clinical parameter determination means (150) for determining at least one clinical parameter based on said processed tracheal pressure values. A corresponding method is also described.

### TECHNICAL FIELD OF THE INVENTION

The present invention relates to the field of medical devices. More particularly, the present invention relates to methods and systems for analysing resuscitation, for example upon intubation of a patient, the invention being not limited thereto.

### BACKGROUND OF THE INVENTION

When a patient, such as a human being or an animal, needs positive pressure ventilation or chest compression (resuscitation), a number of clinical problems may arise.

One known clinical problem is the occurrence of increased intrathoracic pressures during resuscitation. There are numerous case reports of restoration of a spontaneous circulation after cessation of resuscitation efforts. This phenomenon, also referred to as the “Lazarus phenomenon” is mainly explained by trapping of air during ventilation and the presence of “positive end expiratory pressure” (PEEP) resulting in inefficacy or failure of the resuscitation. As trapped air escapes and the positive end expiratory pressure disappears after cessation of the resuscitation, this may allow blood to start flowing to the heart again and therefore result in restoration of circulation even after CPR efforts have been stopped.

Animal studies have also shown that hyperventilation during resuscitation results in decreased coronary perfusion pressure and in excess mortality. In a small clinical observational study of 13 patients with cardiac arrest, high ventilation rates and increased intrathoracic pressures were recorded. Hyperventilation is common during resuscitation. Such findings have resulted in the international recommendation to avoid hyperventilation during resuscitation for cardiac arrest.

Early detection and avoidance of hyperventilation and subsequent increased intrathoracic pressures during resuscitation may be an accurate means for preventing failure of resuscitation and for increasing survival chances and therefore is an important clinical issue.

Another known problem with resuscitation is wrongful intubation. Wrongful intubation into the oesophagus, if detected too late, may result in the death of the patient because of lack of oxygen and ventilation. Wrongful oesophageal intubation is a common problem in emergency situations, both during cardiac arrest and in patients with spontaneous circulation (the latter needing protection of the airway such as in neurotrauma or in cases of respiratory failure).

A variety of methods to detect correct, i.e. tracheal, intubation are known such as for example clinical assessment by looking at chest movements, by auscultation of the chest and of the epigastrium, by assessment of the suction of air through the tube by means of a self-inflating bulb or syringe, by capnography and capnometry, by chest impedance measurements through surface electrodes, etc. None of these techniques are both highly sensitive and specific.

Current state of the art methods to assess quality of resuscitation mainly use impedance measurement of the chest wall and accelerometers placed on the breastbone. The quality of ventilation is often currently addressed by impedance measurements between two electrodes attached to the chest of the victim. This provides reasonable accurate measurements of ventilation frequency and very rough measurements of volume. The quality of chest compression is determined by accelerometers placed on the breastbone of the victim. These provide reasonable accurate measurements of compression frequency and dept.

All these technical solutions to improve the quality and safety of intubation, ventilation and chest compression are in their early stages of clinical application and there is room for improvement.

### SUMMARY OF THE INVENTION

It is an object of embodiments of the present invention to provide alternative methods and systems for analysis of resuscitation. Accurate analysis of resuscitation is an advantage of embodiments according to the present invention. It is an advantage of embodiments according to the present invention that an accurate and quick detection of the position of an endotracheal tube can be determined. It is an advantage of embodiments according to the present invention that accurate detection of the proper position of an endotracheal tube may be obtained, substantially independent of the person who needs to perform the detection. It is an advantage of embodiments according to the present invention that an accurate and quick detection of spontaneous cardiac activity may be obtained.

It is an advantage of embodiments according to the present invention that the system and method can be developed into a standalone device or that it can be incorporated into existing resuscitation monitors and ventilators.

It is an advantage of embodiments according to the present invention that, for some existing monitors, defibrillators or ventilators, the method can be implemented by introducing software without requiring complex additional hardware components and without the need for additional adjuncts such as bulbs, syringes or capnometry equipment. It is for example sufficient that a spare pressure channel is available or can be provided on the monitor, defibrillator or ventilator for allowing receipt of a pressure signal from a pressure sensor in combination with the use of a pressure sensor.

The above objective is accomplished by a method and device according to the present invention.

The present invention relates to a system for analysing resuscitation, the system comprising an input means for receiving or obtaining a plurality of tracheal pressure values over time for tracheal pressure during resuscitation, a tracheal pressure value processing component for processing the obtained tracheal pressure values, and a clinical parameter determination means adapted for determining in real time at least one clinical parameter based on said processed tracheal pressure values.

The present invention also provides a system for analysing resuscitation, the system comprising: a tracheal pressure sensor for receiving or obtaining a plurality of tracheal pressure values over time for tracheal pressure during resuscitation, a tracheal pressure value processor for processing the obtained tracheal pressure values, and a clinical parameter determination means adapted for determining in real time at least one clinical parameter based on said processed tracheal pressure values.

In accordance with some embodiments of the present invention the clinical parameter is not a diagnosis as such nor does it provide or lead to a diagnosis directly. That is, in accordance with some embodiments, the clinical parameter is only information from which relevantly trained personnel could deduce some form of diagnosis however only after an intellectual exercise that involves judgement.

The tracheal pressure sensor for receiving a plurality of tracheal pressure values over time for tracheal pressure during resuscitation, the tracheal pressure value processor for processing the obtained tracheal pressure values, and the clinical parameter determination means adapted for determining in real time at least one clinical parameter based on said processed tracheal pressure values are optionally in some embodiments all ex vivo.

It is an advantage of embodiments according to the present invention that a system is obtained allowing quick and automated detection of appropriate resuscitation using an endotracheal tube.

The tracheal pressure value processing component is a tracheal pressure gradient calculation component for determining at least one tracheal pressure gradient value based on said obtained tracheal pressure values. It is an advantage of embodiments according to the present invention that by using real-time analysis, fast detection of appropriate resuscitation may be obtained.

The tracheal pressure gradient calculation component may be adapted for determining a temporal gradient in tracheal pressure values.

The system may be adapted for analysing resuscitation using an endotracheal intubation tube, wherein the clinical parameter determination means may be adapted for determining whether the intubation tube is positioned oesophageal or tracheal based on said at least one tracheal pressure gradient value. It is an advantage of embodiments according to the present invention that detection of erroneous location of an endotracheal tube can be obtained rapidly after intubation.

The clinical parameter determination means may be adapted for determining whether the tracheal pressure gradient value is higher or lower than a first predetermined value. It is an advantage of embodiments according to the present invention that using at least one gradient value for evaluating may allow to obtain relevant clinical parameters assisting in the assessment of resuscitation.

The clinical parameter determination means may be adapted for evaluating sequential values of the temporal tracheal pressure gradient value.

It is an advantage of embodiments according to the present invention that using such algorithms for evaluating sequential temporal values, the accuracy of detection can be largely improved, thus resulting in the possibility for more accurate resuscitation.

A system according to any of the previous claims, wherein the clinical parameter determination means may be adapted for determining whether spontaneous cardiac activity is present.

It is an advantage of embodiments according to the present invention that detection of spontaneous cardiac activity can be determined rapidly.

The clinical parameter determination means may be adapted for detecting at least two subsequent steps of

a tracheal temporal pressure gradient value higher than a first predetermined value,

followed by a tracheal temporal pressure gradient value with absolute value lower than a second predetermined value,

followed by a high negative temporal tracheal pressure gradient value having an absolute value higher than a third predetermined value.

It is an advantage of embodiments according to the present invention that accurate detection of the location of an endotracheal tube can be obtained.

The system may be adapted for analysing resuscitation using an endotracheal intubation tube, wherein the tracheal pressure gradient calculation component is adapted for determining a spatial gradient in tracheal pressure values based on tracheal pressure values obtained at different positions in an endotracheal intubation tube.

The clinical parameter determination means furthermore may be adapted for determining whether a maximal ventilatory pressure is below a fourth predetermined value. It is an advantage of embodiments according to the present invention that accurate detection of the location of an endotracheal tube can be confirmed.

The clinical parameter determination means furthermore may be adapted for determining a true compression.

The clinical parameter determination means may be adapted for determining whether a temporal pressure gradient value is above a fifth predetermined value, followed by a negative temporal pressure gradient value having an absolute value above a sixth predetermined value and wherein the highest pressure value is above a seventh predetermined value. It is an advantage of embodiments according to the present invention that accurate detection of true chest compressions can be derived.

The system may be adapted for receiving pressure values sensed within an endotracheal intubation tube.

The endotracheal intubation tube may comprise a pressure sensor catheter having a catheter tube filled with air. It is an advantage of embodiments according to the present invention that accurate detection of small variations in intrathoracic pressure can be measured.

The present invention also relates to a method for analysing resuscitation, the method comprising receiving or obtaining a plurality of pressure values over time, processing said obtained tracheal pressure values, and determining in real time at least one clinical parameter based on said processed tracheal pressure values.

In some embodiments the receiving of the plurality of pressure values over time, the processing of said obtained tracheal pressure values, and the determining in real time at least one clinical parameter based on said processed tracheal pressure values are all carried out ex-vivo.

The method furthermore may comprise assessing the resuscitation based on at least one clinical parameter and, if inappropriate, adapting the resuscitation.

The present invention also relates to a monitor, ventilator or defibrillator comprising a system for analysing resuscitation as described above.

The present invention also relates to a computer program product for, when executed on a computer, performing a method of analysing resuscitation as described above.

The present invention furthermore relates to a machine readable data storage device storing the computer program as described above and/or to the transmission thereof over a local or wide area telecommunications network.

It is an advantage of embodiments according to the present invention that, although it requires real-time analysis of a sample pressure signal, it still can be easily integrated in existing equipment already used such as a monitor or a ventilator. It is an advantage that only a pressure sensor needs to be added or integrated in the system. It is an advantage of embodiments according to the present invention that no bulb, syringe or capnometry is necessary. It is an advantage of embodiments according to the present invention that real-time analysis of the pressure signal may be performed, as this allows for direct adjustment of the intervention taking place, thus increasing the chances of a patient to be resuscitated successfully and to survive.

It is an advantage of embodiments according to the present invention that analysis can be performed in an automated and/or automatic way. The latter reduces the risk on errors, as less human intervention may be required.

It is an advantage of embodiments according to the present invention that the endotracheal pressure sensor catheters can be air-filled. It thereby is an advantage that small pressure differences induced by compression or spontaneous heart activity can be detected. The endotracheal pressure thereby may be used as surrogate for intrathoracic pressure.

It is an advantage of embodiments according to the present invention that the methods and systems can be used both for patients in cardiac arrest and for patients without cardiac arrest.

It is an advantage of embodiments according to the present invention that the effect of chest compression is measured rather than the compression depth itself.

It is an advantage of embodiments that the systems and methods use gradients in the intrathoracic pressure values for analysing clinical parameters, allowing to derive important clinical parameters for analysing the resuscitation and thus allowing accurate assessment of the resuscitation.

Particular and preferred aspects of the invention are set out in the accompanying independent and dependent claims. Features from the dependent claims may be combined with features of the independent claims and with features of other dependent claims as appropriate and not merely as explicitly set out in the claims.

The teachings of the present invention permit the design of improved methods for resuscitation.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of a system for analysing resuscitation according to an embodiment of the present invention.

FIG. 2 is a schematic representation of a flow chart of the algorithm that may be used for deriving information for the analysis of resuscitation according to an embodiment of the present invention.

FIG. 3 is a schematic representation of an exemplary tracheal ventilation pressure curve for oral intubation and mechanical ventilation as can be used in an embodiment according to the present invention.

FIGS. 4A and 4B are schematic representations of an exemplary tracheal ventilation pressure curve on the one hand (FIG. 4A) and an exemplary oesophageal ventilation pressure curve on the other hand (FIG. 4B), as can be used in embodiments according to the present invention.

FIG. 5a, FIG. 5b, FIG. 5c and FIG. 5d illustrate pressure curves for a distal measurement point and a proximal measurement point in case of tracheal intubation (FIG. 5a and FIG. 5b) and in case of oesophageal intubation (FIG. 5c and FIG. 5d) as can be obtained according to embodiments of the present invention.

FIG. 6 is a schematic representation of a computing device as can be used for performing processing steps in a method for analysing resuscitation according to an embodiment of the present invention.

FIG. 7 is a schematic flow chart illustrating an algorithm for determining a clinical relevant parameter, according to an embodiment of the present invention.

FIG. 8a, FIG. 8b and FIG. 8c illustrate output windows displaying the received pressure curves and derived clinical parameters according to an embodiment of the present invention (FIG. 8a) as well as output windows for insufflation analysis for a mechanical ventilation without CPR (FIG. 8b) and with CPR (FIG. 8c) as can be obtained according to embodiments of the present invention.

In the different figures, the same reference signs refer to the same or analogous elements.

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practised without these specific details. In other instances, well-known methods, structures and techniques have not been shown in detail in order not to obscure an understanding of this description.

In a first aspect, the present invention relates to a method for or a system adapted for analysing resuscitation. In embodiments according to the present invention the system or corresponding method may for example more particularly be adapted for analysing intrathoracic pressure during resuscitation. The system may be adapted for providing analysed intrathoracic pressure data to the user, e.g. rescuer. Alternatively or in addition thereto, the system may be adapted for providing an indication of a status of the patient or a status or quality of the resuscitation, i.e. provide an assessment of the patient or the resuscitation based on the obtained analysis results. Resuscitation thereby typically may comprise external chest compression and non-invasive ventilation. The system and/or method may be part of or be used in combination with a monitor, ventilator or defibrillator. A ventilator may for example be a mechanical ventilator as well as with a device for manual ventilation e.g. a self-inflating bag device. The ventilator advantageously is autonomous.

According to embodiments of the present invention, the system and/or method is adapted for receiving or obtaining measured tracheal pressure values for the patient. In some embodiments, the measured tracheal pressure values thereby advantageously are obtained at a distal end of the endotracheal tube, i.e. for example via a catheter inserted in the endotracheal tube intubated in the patient. Such signals may advantageously provide information regarding certain clinical parameters, not or less available in pressure signals captured at the proximal end of the endotracheal tube. Alternatively, the measured values may be obtained further away from the distal end of the endotracheal tube, e.g. at the proximal end of the endotracheal tube. In some embodiments, measured tracheal pressure values may be obtained at least two different positions in the endotracheal tube. The measured tracheal pressure values may for example be obtained at the distal end of the endotracheal tube and at the proximal end of the endotracheal tube. In some embodiments, combinations of such values may be used for deriving certain clinical parameters. In some embodiments, the measured tracheal pressure values may be measured when a supraglottic device is used or with a self inflating bag device with a mask, i.e. some embodiments of the present invention relate to resuscitation without endotracheal tube. As described further below, receiving the measured tracheal pressure values may be receiving at an input channel of the system tracheal pressure values measured with a component not part of the system. The receiving measured tracheal pressure values than results in receiving corresponding data.

The system and/or method furthermore may be adapted for determining from said measured tracheal pressure values a tracheal pressure gradient. The tracheal pressure gradient may for example be a gradient of the measured tracheal pressure values, a gradient on smoothed tracheal pressure values or a gradient of the tracheal pressure values modified by subtracting an average tracheal pressure value determined in a moving window. The pressure gradient may be a temporal gradient of the measured tracheal pressure values, although embodiments of the present invention are not limited thereto and a spatial gradient of such pressure values also is envisaged. Embodiments of the present invention furthermore are adapted for determining in real-time at least one clinical parameter based on the tracheal pressure values obtained. The clinical parameters may be a variety of clinical parameters such as for example the correctness of intubation including the location of the tube being intratracheal or oesophageal, or for example the quality of ventilation, including the occurrence of spontaneous ventilation and restoration of spontaneous circulation, i.e. spontaneous cardiac activity, the quality of obtained intrathoracic pressure, etc. In some embodiments of the present invention, the system and/or method thus may be adapted for determining the difference between oesophageal intubation and tracheal intubation. The latter may be advantageous as often erroneously oesophageal intubation occurs, which may have severe consequences for the patient if realised or recognised late, e.g. it may result in hypoxia, cerebral damage, dead. Discrimination between oesophageal intubation and tracheal intubation may in embodiments according to the present invention be based on ventilation pressure patterns. It is an advantage of some embodiments according to the present invention that detection of oesophageal intubation can be performed very accurately and/or early during the resuscitation process. The sensitivity and specificity of detecting oesophageal intubation can for example be improved significantly using a detection algorithm based on pressure gradients. In another embodiment, the methods and/or systems provide an indication of the intrathoracic pressures that occur, e.g. an indication or warning when an increased intrathoracic pressure occurs. According to examples of some embodiments of the present invention, the system may be adapted for providing information regarding restoration of spontaneous ventilation and restoration of spontaneous circulation, i.e. spontaneous cardiac activity. In one embodiment, the system may be adapted for indicating whether a proper chest compression rate is achieved by the rescuer. In one embodiment, the methods and/or systems additionally may provide an indication of the ventilation frequency, e.g. including an indication or warning when the ventilation frequency is too high or too low. In another embodiment, the methods and/or systems may provide an indication of a wrong ventilation frequency and high pressures occurring. The system may be adapted in a hardware-based manner as well as in a software-based manner. In accordance with some embodiments of the present invention the clinical parameter is a not a diagnosis as such nor does it provide or lead to a diagnosis directly. That is, in accordance with some embodiments, the clinical parameter is only information from which relevantly trained personnel could obtain relevant medical conclusions however only after an intellectual exercise that involves judgement.

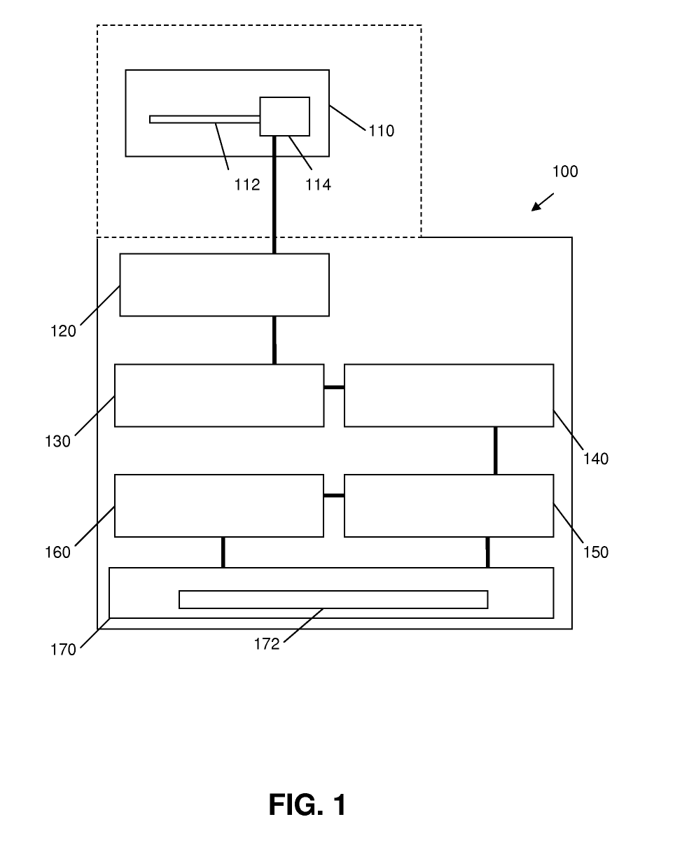

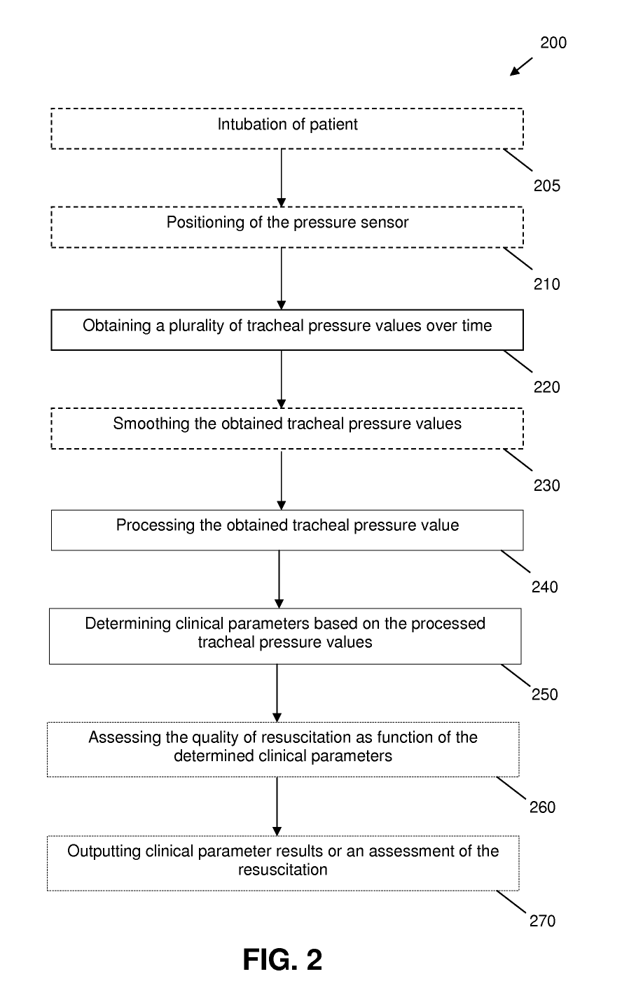

By way of illustration, the present invention not being limited thereto, an exemplary system and/or method according to an embodiment of the present invention is described. The exemplary system is shown with reference to FIG. 1, indicating standard and optional components of a system for analysing resuscitation. The exemplary method is shown with reference to FIG. 2, indicating standard and optional steps of a method

The system 100 according to an embodiment of the present invention may be provided with at least one pressure sensor 110 or it may be adapted to receive information from at least one pressure sensor 110. The at least one pressure sensor 110 may be any suitable pressure sensor for measuring pressure, advantageously a pressure sensor for measuring pressure at the distal end of the endotracheal tube. Alternatively or in addition thereto, a pressure sensor 110 also may be adapted for measuring pressure e.g. when using a supraglottic device or a self inflating bag device with mask.

The at least one pressure sensor may be adapted for positioning a sensing part at the distal end of the endotracheal tube, e.g. close to the distal end of the endotracheal tube such as e.g. at about 2 cm from the distal end of the endotracheal tube of the patient. Alternatively, the at least one pressure sensor may be adapted for positioning a sensing part at the proximal end of the endotracheal tube. In some embodiments, tracheal pressure values may be determined at least two different positions in the endotracheal tube. The latter provides the advantage that a spatial tracheal pressure gradient value can be determined, which may allow determination of clinical parameters in an accurate way. The at least one pressure sensor may be adapted for being inserted in the tube used when intubating the patient. One example of pressure sensor 110 that can be used is a catheter pressure sensor. The proximal end of such a catheter may optionally be connected to a bacterial filter (Intersurgical) and may be further connected to a pressure transducer. The catheter pressure sensor may comprise an air filled catheter 112, allowing to detect small variations in pressure. Pressure may be measured by transfer of a pressure signal sensed in catheter 112 to a pressure transducer 114, allowing to transfer the sensed signal into data. If detected in an analogue mode, the pressure data may be digitized. The pressure signal may, if appropriate intubation is performed, be a tracheal pressure signal. The obtained signal then is the sum of the pressure generated by positive pressure ventilation, chest compression, spontaneous breathing and spontaneous cardiac activity. The corresponding method 200 may optionally be adapted for measuring or assessing tracheal pressure signals using a pressure sensor as described above. The method thus may comprise intubating 205 the patient with an endotracheal tube and positioning 210 a pressure sensor for sensing intratracheal pressure or alternatively, it may be limited to a method initiated by obtaining pressure sensor data.

According to embodiments of the present invention, the system 100 and/or method 200 is adapted for receiving or obtaining 220 measured tracheal pressure values. These samples may be received over any suitable telecommunications channel. For example, these values may be obtained via a wireless or a wired communication channel. The measured tracheal pressure values may be representative for a plurality of samples of the pressure over time. Advantageously, the sampling rate may for example be at least 10 Hz, more advantageously at least 25 Hz, even more advantageously at least 50 Hz. The latter results in a number of pressure values Pₓ at sampling points x, representative of time. The measured tracheal pressure values may be digitized or may be received in digitized form. The system may comprise an input means 120, also referred to as input port, for obtaining a plurality of tracheal pressure values over time. The input means 120 thereby may be adapted for receiving the pressure data directly from the pressure sensor 110 by performing the measurement act, whereby the system does not need to include the measurement equipment but only needs to be adapted for receiving the tracheal pressure data. Similarly, the method does not need to include the measurement act but only needs to be adapted for receiving as data input the tracheal pressure data.

The system 100 and/or method 200 furthermore is adapted for processing the obtained measured tracheal pressure values. Processing may include amplifying the signals using a suitable amplifier, such as for e.g. a Wheatstone Bridge amplifier. Advantageously, amplification is performed for each channel where tracheal pressure values are obtained. The amplifiers may be selected such that the range of amplification corresponds with the range of measured values, e.g. between −100 mbar and 100 mbar. The system 100 therefore may be adapted in hardware or in software. The system 100 may for example be equipped with processing capacity for performing the processing and may be programmed for performing the processing according to a predetermined algorithm, using a neural network or according to predetermined rules. The system 100 may be adapted for performing the receipt and the processing of the measured tracheal pressure values in an automated and/or automatic way. The processing may be performed in one or more central processors or may be performed in dedicated processing components. In the following description different components for performing the different processing steps will be indicated, but it will be clear to the person skilled in the art that the processing may be performed by the same processor. The processing tasks may be controlled by different software instructions, e.g. different steps in an algorithm. Similarly, intermediate as well as end results may be stored in one or a plurality of memories. Although in the following a single memory is described for storing intermediate and final results, the latter may be split up into several memories. The processing may be performed using a predetermined algorithm, allowing decomposition of the measured pressure signal in the individual contributions. Embodiments of the present invention are adapted for determining in real time at least one clinical parameter based on processing the obtained tracheal pressure values. The processing of tracheal pressure values may allow assisting in clinical assessment during resuscitation. As soon as a cycle of ventilation and/or compression has taken place, the clinical parameters can be determined substantially in real-time.

In a first optional processing step, smoothing 230 of the obtained measured tracheal pressure values may be performed. The system thus may be adapted for smoothing 230 the obtained measured tracheal pressure values, e.g. it may comprise a smoothing component 130 for smoothing. The smoothing component 130 may be software-based or may be dedicated hardware or a combination of software and hardware. The smoothing 230 may be performed to compensate for high frequency artefacts. Smoothing 230 may be performed by determining the mean pressure over a moving time-window of the measured pressure values and determining a smoothed tracheal pressure value there from. In one example, the time-window over which such averaging may be performed may be 150 milliseconds. In this way, the sampled tracheal pressure values may be transformed in a set of new smoothed tracheal pressure values by replacing every sampled value by its average in a time-window surrounding the sampled value. The latter may for example be obtained according to following algorithm, i.e.

For a number z of samples Pₓ

P₁, P₂, . . . P_{z}

the corresponding smoothed tracheal pressure value Sₓ can be determined by

$${S}_{x} = \frac{{\sum }_{i = - n + 1}^{0} {P}_{( x + i )}}{n}$$

wherein n is the number of samples in the moving time-window. For the initial n samples, the number of samples used for the smoothing may be gradually increased from 1 to n, or the initial values may be discarded. This smoothed waveform may be used for subsequent calculation of one, more or all of the ventilatory parameters of interest. Alternatively the non-smoothed measured pressure values may be used for further processing.

In a further processing step, the tracheal pressure values may be processed 240. The processing may comprise determining at least one tracheal pressure gradient value. Determining at least one tracheal pressure gradient value may be based on the smoothed tracheal pressure values or based on the measured tracheal pressure values without smoothing. Other processing also may be performed as described below. The system thus may be adapted for processing the tracheal pressure values, it may e.g. comprise a tracheal pressure value processing component 140 for processing the tracheal pressure values. The tracheal pressure value processing component 140 may be a tracheal pressure gradient calculation component for determining a tracheal pressure gradient value. The gradient thereby may be a temporal or spatial gradient. The temporal gradient, which may be expressed as dP/dt, expresses a variation of the pressure over time, whereas the spatial gradient, which may be expressed as dP/ds, expresses a variation of the pressure between two different locations. The tracheal pressure processing component 140 may be software-based or may be dedicated hardware or a combination of software and hardware.

The tracheal pressure gradient may be a temporal tracheal pressure gradient and/or a spatial tracheal pressure gradient. The tracheal pressure gradient may be a temporal tracheal pressure gradient determined based on a derivative over time of the tracheal pressure values. The temporal gradient in tracheal pressure may be determined by determining a derivative of the pressure waveform constituted by the tracheal pressure values, optionally the smoothed tracheal pressure values. In one embodiment, the latter is performed by determining the gradient of the ventilatory pressure in a time window around the sample or smoothed sample. In one example, the time window over which determination of the gradient may be performed may be 150 milliseconds. For samples Pₓ or the smoothed sample Sₓ the gradient value Gₓ may be determined as

$${G}_{x} = ( {P}_{x} - {P}_{( x - n )} ) * \frac{R}{n}$$

$$respectively$$

$${G}_{x} = ( {S}_{x} - {S}_{( x - n )} ) * \frac{R}{n}$$

whereby R is the sampling rate, n is the number of samples in the time window. Gₓ thereby is expressed in pressure per time unit.

According to embodiments of the present invention, the method and/or system furthermore is adapted for determining 250 at least one clinical parameter based on at least a pressure gradient value. The system thus may be adapted for determining at least one clinical parameter based on at least a pressure gradient value and therefore may comprise a clinical parameter determination component 150. The clinical parameter determination component 150 may be software-based or may be dedicated hardware or a combination of software and hardware. As already indicated above a plurality of clinical parameters may be determined based on at least a pressure gradient value obtained in the previous step. By way of illustration, some examples are provided, the invention not being limited thereto.

In a first particular example, the system, more particularly the clinical parameter determination component 150, may be adapted for determining detection of the location of an intubated tube, i.e. oesophageal or intratracheal intubation, based on at least one pressure gradient value. When oesophageal intubation is performed, it has been found that the pressure profile typically consists of a fast increase of the sampled pressure or smoothed sampled pressure, thereafter switching to a plateau pressure, followed by a fast decrease of the sampled pressure or smoothed sampled pressure. It furthermore has been found that in oesophageal ventilation, the maximal ventilatory pressure is never above a relatively low cut-off value even if forceful ventilation is applied by the rescuer. Since the volume of air that can be insufflated into the oesophagus is much lower, the flow through the tube is relative low at any pressure. Consequently, the pressure gradient between the distal and proximal measuring point is lower than for tracheal ventilation.

On the other hand when tracheal intubation, as typically is required, is performed, insufflation of air through the endotracheal tube induces a flow of air into the lungs. Because of the capacity of the lungs to accept a significant volume of air, the flow or air through the tube (e.g. expressed in ml/s) results in a clear pressure gradient between the proximal and the distal measurement point, if two points are used for measuring tracheal pressure or receiving info thereof. Furthermore at expiration, since an important volume of air can be exhaled when the insufflation pressure is released and the patient is allowed to exhale, the pressure at the proximal measuring point drops immediately, while the pressure at the distal measuring point only drops slowly due to the important volume of air that needs to flow through the tube. Again a pressure gradient develops between the two measuring points. Because of the lower compliance of the oesophagus compared to the lungs, the increase in pressure at the proximal and even more the distal measuring point is significantly less steep in tracheal than in oesophageal ventilation. The gradient G of the pressure signal thus may be significantly lower than the high absolute values obtained during oesophageal intubation and higher than the gradient value during the plateau in the oesophageal intubation. Also at expiration the absolute amplitude of the gradient G of the pressure signal is much lower. It also has been found that the maximal ventilatory pressure in tracheal ventilation is much higher than in oesophageal ventilation, even though the gradient G of the pressure is significantly lower. The difference in compliance between the lungs and the oesophagus thus results in very significant differences in the characteristics of the pressure gradient over time of the endotracheal pressures and of the pressure gradient between two different measuring points at a given time.

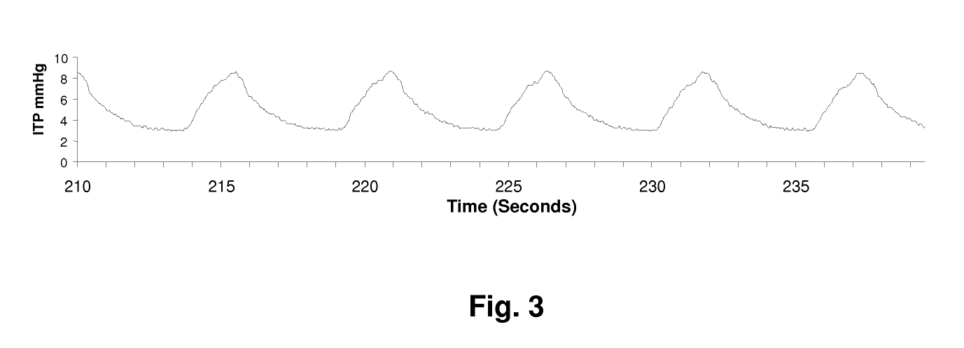

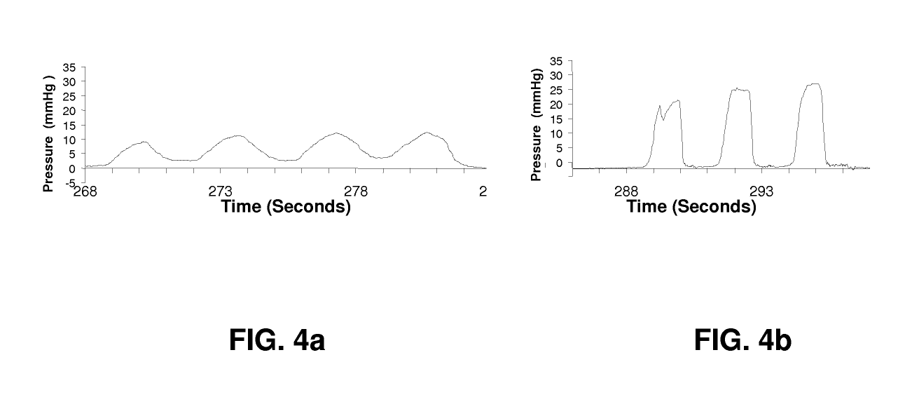

By way of illustration, FIG. 3 and FIG. 4a and FIG. 4b illustrate intrathoracic pressure curves as obtained during resuscitation. FIG. 3 thereby is a schematic representation of an exemplary tracheal ventilation pressure curve for oral intubation and mechanical ventilation. In FIG. 4a and FIG. 4b schematic representations of an exemplary tracheal ventilation pressure curve on the one hand (FIG. 4a) and an exemplary oesophageal ventilation pressure curve on the other hand (FIG. 4b) are shown for manual ventilation, indicating the different pressure behaviour resulting in the different pressure gradient behaviour as described above.

In some embodiments, the system may be adapted for determining intubation, e.g. detecting oesophageal intubation based on the tracheal pressure gradient value being higher than a predetermined value. The predetermined value may depend on a plurality of factors which may be provided as input at an input unit of the system. Potential patient related factors may be the compliance of the chest, the compliance of the lungs, the performance of chest compression, etc. These may be taken into account, depending on their degree of interference. If the pressure gradient value is at any time or during a period of the cycle higher than a predetermined value, the system may be adapted for providing a warning or alarm signal, indicative of a significant chance of oesophageal intubation instead of tracheal intubation. In another embodiment, the system may be adapted for determining the location of the tube by providing a qualitative evaluation of the sequential values of the gradient G, allowing for example to detect a high gradient value, followed by a low or substantially zero gradient value, thereafter followed by a high negative gradient value. This sequence or e.g. two subsequent steps therein, may be used as indication for oesophageal intubation. In one embodiment, the system may be adapted for determining intubation, e.g. detecting oesophageal intubation, based on the maximal ventilatory pressure that is measured, in addition to the pressure gradient value used. The system may be adapted for providing an indication of the maximal ventilatory pressure that is measured. If the maximal ventilatory pressure is below a relatively low cut-off value, the system may be adapted for providing a warning or alarm signal, indicative of a significant chance of oesophageal intubation instead of tracheal intubation. In this embodiment, both information regarding the gradient G of the pressure signal and information regarding the maximal ventilatory pressure may be used to determine the chance of oesophageal intubation or tracheal intubation.

It has been found that using the pressure curves obtained during the initial ventilation cycles, e.g. during the first four ventilation cycles, correctness of the intubation can be determined, i.e. distinction can be made between tracheal intubation or oesophageal intubation. It is an advantage of embodiments of the present invention that sampling the pressure signal generated by the ventilations can be performed as soon as intubation has been performed, thus allowing to quickly distinguish between oesophageal and tracheal intubation. The latter can be indicated, e.g. using an alarm or warning signal in any suitable way, e.g. using a green light when tracheal intubation is obtained and using a red light when oesophageal intubation is obtained. According to embodiments of the present invention, the system thus may provide confirmation of the localization of the tube being intratracheal or oesophageal upon intubation. This information will allow the health care provider to establish correct intubation or to remove and replace the tube.

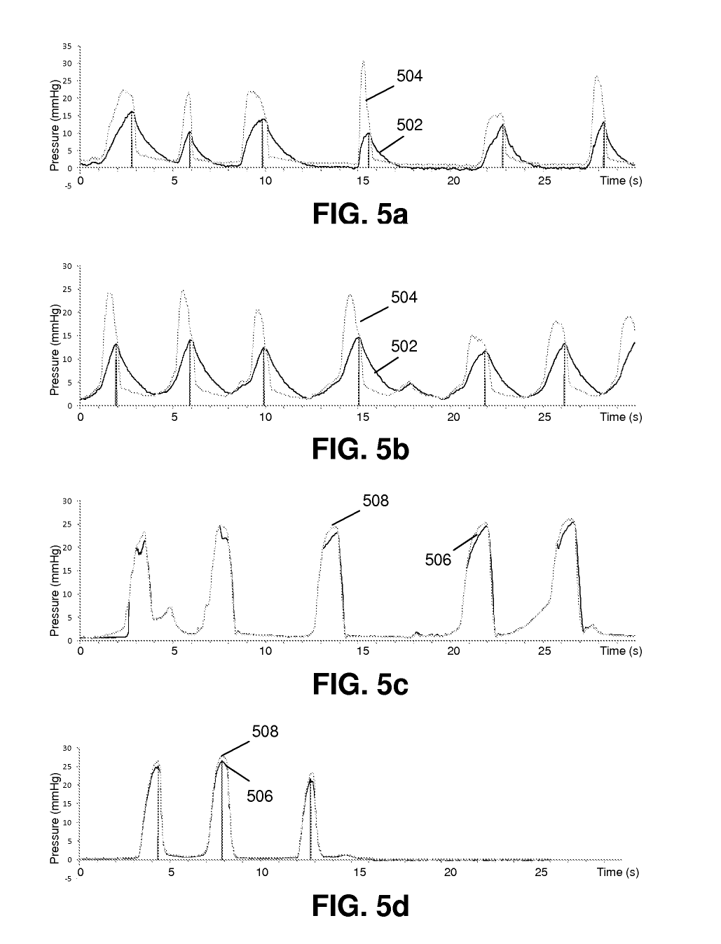

Further examples of tracheal and oesophageal manual ventilation in humans are shown in FIG. 5a, FIG. 5b, FIG. 5c and FIG. 5d, whereby FIG. 5a and FIG. 5b illustrates the pressure at a distal 502 and proximal 504 measurement point to the lungs for two different patients for tracheal intubation, and FIG. 5c and FIG. 5d illustrates the pressure at a distal 506 and proximal 508 measurement point to the lungs for two different patients for oesophageal intubation.

In another example, the gradient G may be used for determining the onset and release of chest compressions. When the gradient is above a predetermined value, e.g. above a predetermined cut-off value, a true compression may be suspected. If a gradient with a negative value of at least a predetermined value is subsequently detected within 500 ms and the highest pressure value between both gradient values is above a predetermined value, a true compression may be confirmed. The highest pressure value may be referred to as peak pressure. The system may be adapted to use the time between the two or some of the last maximal pressure values for determining a rate of chest compression. The system may be adapted for providing a notification when the determined chest compression rate is too high or too low. The lowest pressure value Pₓ in the 250 ms after the minimal gradient value Gₓ is the minimal pressure. Ideally, to achieve optimal venous return and blood flow to the heart, this value should be zero or negative. The system may be adapted for providing a warning or alarm notification if the minimal pressure does not return to baseline. Evaluation may be performed during several subsequent compressions. The latter may for example occur when there is incomplete release of compression. The system also may be adapted for determining a mean pressure generated by a chest compression. The latter may be determined by

$${P}_{m} = \frac{{\sum }_{i = {T}_{1}}^{{T}_{2}} {P}_{( i )}}{{T}_{2} - {T}_{1} + 1}$$

with point T₁ and T₂ being the time point of maximal Gₓ values of the two last compressions. The system furthermore may be adapted for determining a difference between de Peak Pressure and the Minimal Pressure, referred to as ΔP. If the amplitude of ΔP is too low, a warning or alarm notification may be provided.

In another particular example, the system is adapted for detecting spontaneous circulation. Spontaneous circulation may be evaluated based on a pulse pressure PP determined as follows: With M₁ being the minimal pressure value in a time span of 200 ms before the positive gradient value is obtained and M₂ being the minimal value in a time span of 200 ms after the negative gradient value, the minimum pressure can be determined as

$${P}_{min} = \frac{{M}_{1} + {M}_{2}}{2}$$

The peak pressure Pₚₑₐₖ can be determined as the highest pressure value between the positive gradient and the negative gradient.

The pulse pressure PP then is defined as

PP=Pₚₑₐₖ−Pₘᵢₙ

If the pulse pressure is higher than a minimal predetermined value, spontaneous circulation may be confirmed. Advantageously, also a gradient higher than a minimum value and a positive gradient value followed by a negative gradient value of minimal absolute value within 200 ms are factors pointing to spontaneous circulation. The combination of the above three aspects (pulse pressure, gradient value and subsequent positive and negative gradient) may allow confirmation of spontaneous circulation.

The tracheal pressure gradient may be a spatial tracheal pressure gradient based on tracheal pressure values determined at different positions in the endotracheal tube. The behaviour of the tracheal pressure values at the different positions may allow to derive the origin of pressure built up. If for example an abrupt pressure pulse is measured at the distal end of the endotracheal tube and a smaller pressure pulse is measured at the proximal end of the endotracheal tube, the tracheal pressure signal is more likely representative of a chest compression. If for example a weaker pressure pulse is measured at the distal end than the pressure pulse measured at the proximal end of the endotracheal tube, the tracheal pressure signal is more likely representative of a ventilation.

The method and/or system may be adapted for also determining further clinical parameters. The system therefore may comprise a additional parameter determination component 180. The system and/or method may for example be adapted for determining the mean pressure Mₓ at sample point x by averaging the sampled pressure values or the smoothed values thereof over a large time window, e.g. over a time window of 5000 ms. In further embodiments, this value may be used for determining whether the sampled pressure value or the smoothed sampled pressure value is below or above the mean pressure and the inversion point, for determining the highest value H of the sampled pressure values or the smoothed sample pressure values and/or for determining the lowest value L of the sampled pressure values or the smoothed sampled pressure values. Both timing and value of the maximal and minimal ventilatory pressure can be derived. Evaluation of the sign of ((P_{X} or S_{X})−Mₓ) may allow to determine whether the sampled or smoothed sampled pressure is below or above mean pressure. Determination when ((Pₓ or Sₓ)−Mₓ) equals zero may allow to determine inversion points. Calculation of the mean pressure may be performed continuously, using a moving window.

In one embodiment, the system optionally may be adapted for diagnosing a ventilation cycle, with a true sign inversion, if the highest sampled, optionally smoothed, pressure value minus the lowest sampled, optionally smoothed, pressure value is larger than a predetermined value, e.g. larger than 5 cmH₂O.

In one embodiment, the system optionally may be adapted for determining the ventilation frequency based on the time between two sub-sequent peak ventilatory pressures. In another embodiment, the system may be adapted for determining within every ventilation cycle, the fraction of the time during which the ventilatory pressure is higher than a certain value. The obtained fraction may be used as signalling function, e.g. when the fraction is higher than a certain value an alarm signal may be provided. In yet another embodiment, the system may be adapted for determining whether a minimal ventilatory pressure is higher than a certain value. The latter may be used as signalling function, e.g. when the minimal ventilatory pressure is higher than a certain value, an alarm signal may be provided. This would signify the presence of PEEP and a risk of decreased venous return and lower efficacy of the chest compressions. The system may be adapted for providing an alarm signal if the ventilation frequency is or is repeatedly higher or lower than a certain value. The system may be adapted to provide an alarm signal if the maximal ventilatory pressure is higher than a certain value. In one embodiment, the system may be adapted for providing a notification of spontaneous respiration if a negative ventilatory pressure below a certain value is detected.

In a further step, the method and/or system advantageously may be adapted for assessing 200 the quality of the resuscitation based on the determined clinical parameters. Such an assessment may be performed in an automated and/or automatic way and results may be outputted or it may be performed by the user based on outputted determined clinical parameter results. The system 100 may be adapted with an assessment component 160 for assessing the resuscitation based on the determined clinical parameter results. The assessment component 160 may be software-based or may be dedicated hardware or a combination of software and hardware.

The method and/or system therefore advantageously also may be adapted for optionally generating 270 an output representative of the assessment of at least one clinical parameter or a related, e.g. physical, condition or an assessment of the resuscitation. The system therefore may comprise an output generating means 170. The latter may for example be a printer, plotter, speaker, display, lighting system, etc. The output may allow the user, e.g. rescuer, to maintain, adjust or stop his action. The output may be generated in a plurality of ways, the invention not being limited thereby. It may be data outputted on a plotter, printer or screen, it may be data outputted as sound signal or voice signal, it may be data visualised by colour, e.g. via coloured lamps, etc. or a combination of these. The system may be equipped with a user interface 172 for example allowing the user to select output information that he requires.

In some embodiments, the pressure data and/or clinical parameters may be stored in a memory, e.g. a memory of the system. The data thus can be recalled and used for debriefing and/or post-intervention evaluation of the resuscitation. Such information can be used for educational purposes or as a report of the resuscitation for medico-legal purposes.

The generated output may have a signalling or warning function. An often used way of generating output, the invention not being limited hereto, is activating a green light if the clinical parameter and/or the corresponding status of the patient or of the resuscitation is acceptable and providing a red light and/or sound signal if the clinical parameter and/or the corresponding status of the patient or of the resuscitation is not acceptable. If the system is part of a monitor, ventilator or defibrillator, outputting of information also may be performed through a single output system used by other components of the monitor, ventilator or defibrillator.

In order to further improve the information obtained with the system, some embodiments of the present invention comprise a system as described above, whereby the system furthermore is adapted with a detector for other signals that may be assisting in assessing clinical parameters, such as for example detection of ECG signals, detection of oxygen saturation, impedance measurements, accelerometric assessment of heart compression, etc. Combining of ECG signals with intrathoracic pressure level information according to an embodiment of the present invention may provide more accurate information regarding spontaneous cardiac activity and spontaneous respiration and thus enhancing the quality of the information. Combining the signals may allow further optimisation of decomposition of intrathoracic pressure values in its components. For example, appearance of a peak in the intrathoracic pressure systematically following the R-wave on an ECG indicates a higher probability of there being a true spontaneous cardiac compression than conclusions drawn when the ECG-information is absent. One possible example of such a detection is given by averaging several loops of the cardiac cycle by using the R-wave as reference starting point of the cycle and then averaging the intrathoracic pressure. Random artefacts should disappear in the averaged signal, while a systematic peak in the intrathoracic pressure would become more evident. The combined signals also may be outputted.

The system according to embodiments of the present invention may be incorporated in existing ventilators or monitors. It thereby is an advantage that the system may be provided in software, so that implementation of the system can be performed relatively easy by installing software on existing systems. The ventilators or monitors further should be provided with a pressure sensor, which can be easily integrated in existing ventilators or monitors The system may be part of a portable monitor, defibrillator and/or ventilator. Alternatively, the system may be a separate device comprising or connectable to a pressure sensor.

It is an advantage of embodiments according to the present invention that one or more of the following data can be obtained: percentage positive pressure over total CPR time, positive end expiratory pressure, detection of spontaneous breathing, detection of spontaneous cardiac activity, incomplete release of compression, quality of intubation, mean and peak ventilation pressure, artificial ventilation frequency, rate of chest compression, mean and peak pressures generated by chest compression, ventilation and chest compression pauses, change of rescuers (by detecting a sudden change in pressure pattern) etc, both lists not being limiting.

In a second aspect, the present invention relates to a monitor, ventilator or defibrillator for providing resuscitation to a patient in need. The monitor, ventilator or defibrillator according to embodiments of the present invention comprises conventional components for allowing ventilation and/or defibrillation, but furthermore comprises a system for assessing the resuscitation as set out in the first aspect. The system may comprise the same features and advantages as set out above.

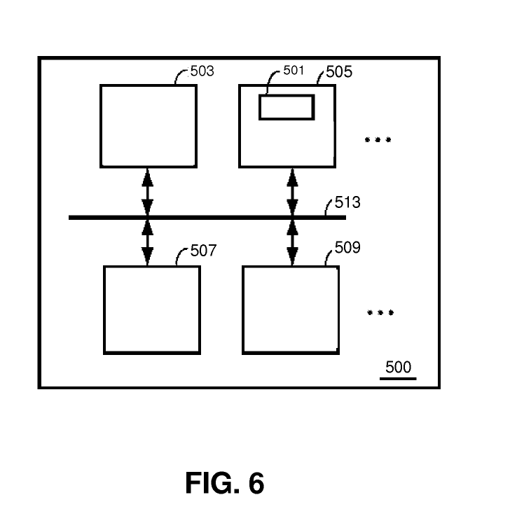

In a third aspect, the present invention relates to a processing system wherein the method or system for assessment of resuscitation as described in embodiments of the previous aspects are implemented in a software based manner. FIG. 5 shows one configuration of a processing system 500 that includes at least one programmable processor 503 coupled to a memory subsystem 505 that includes at least one form of memory, e.g., RAM, ROM, and so forth. It is to be noted that the processor 503 or processors may be a general purpose, or a special purpose processor, and may be for inclusion in a device, e.g., a chip that has other components that perform other functions. Thus, one or more aspects of embodiments of the present invention can be implemented in digital electronic circuitry, or in computer hardware, firmware, software, or in combinations of them. The processing system may include a storage subsystem 507 that has at least one disk drive and/or CD-ROM drive and/or DVD drive. In some implementations, a display system, a keyboard, and a pointing device may be included as part of a user interface subsystem 509 to provide for a user to manually input information. Ports for inputting and outputting data also may be included. More elements such as network connections, interfaces to various devices, and so forth, may be included, but are not illustrated in FIG. 6. The various elements of the processing system 500 may be coupled in various ways, including via a bus subsystem 513 shown in FIG. 6 for simplicity as a single bus, but will be understood to those in the art to include a system of at least one bus. The memory of the memory subsystem 505 may at some time hold part or all (in either case shown as 511) of a set of instructions that when executed on the processing system 500 implement the steps of the method embodiments described herein. Thus, while a processing system 500 such as shown in FIG. 6 is prior art, a system that includes the instructions to implement aspects of the methods for assessing resuscitation is not prior art, and therefore FIG. 6 is not labelled as prior art.

The present invention also includes a computer program product which provides the functionality of any of the methods according to the present invention when executed on a computing device. Such computer program product can be tangibly embodied in a carrier medium carrying machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fibre optics, including the wires that comprise a bus within a computer.

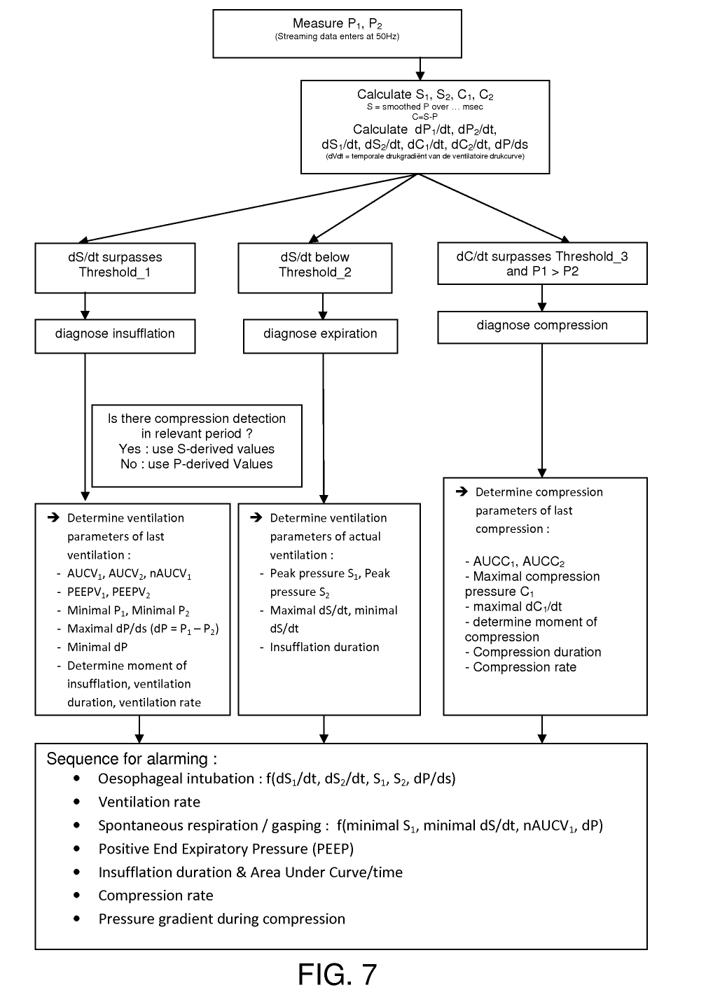

By way of illustration, embodiments of the present invention not being limited thereby, an example of a algorithm that may be used in a system or method as described in the first or second aspect, or in a processing system or computer program product as described in the third aspect, is illustrated in FIG. 7 by way of flow chart 600.

In a first step 610, measurement or receipt of tracheal pressure data is indicated. In the current exemplary algorithm, tracheal pressure values are obtained at two different positions, in this example illustrated by P₁ and P₂, embodiments of the present invention not being limited thereto, so measurements also could be performed at a single location or at more than 2 positions. In the present example P₁ expresses the pressure in the distal end of the endotracheal tube, i.e. used for sensing closer to the lungs, and P₂ expresses the pressure in the proximal end of the endotracheal tube, i.e. used for sensing further away from the lungs. Such values typically may be expressed in mbar. Measurement data typically may be obtained for different moments in time. The data typically may be obtained as streaming data, advantageously e.g. at a frequency sufficiently high to evaluate shape of the signal or the shape of a differential value thereof.

In a second step 620, at least a gradient based on the tracheal pressure value as function of time or position is determined. This may be one of the pressure gradients as described below. The number of parameters that can be calculated may be large. Advantageously following parameters can be calculated:
A ventilatory pressure value S based on the tracheal pressure, obtained by smoothing the tracheal pressure values obtained in a given time window. A series of data may be obtained by using a moving time window for the integration. S₁ and S₂ in the present example thus correspond with smoothed versions of P₁ and P₂ respectively. The smoothed values reflect the ventilatory pressure.
A compression pressure value C based on the tracheal pressure, obtained by subtracting the smoothed tracheal pressure value from the received tracheal pressure value, i.e. C=P−S, resulting in a modified tracheal pressure value reflecting the additional pressure generated by the compressions. In the present example modified pressure values C₁ and C₂ can be determined based on the received tracheal pressure values P₁ and P₂ respectively and on the smoothed tracheal pressure values S₁ and S₂ respectively.
A pressure gradient over time for the received tracheal pressure values, indicated as dP/dt. For the different tracheal pressure values, this can be indicated as dP₁/dt and dP₂/dt respectively.
A pressure gradient over time for the ventilatory pressure values S, indicated as dS/dt, indicating the pressure gradient over time of the ventilation pressure curve. For the different ventilatory pressure values, this can be indicated as dS₁/dt and dS₂/dt respectively.
A pressure gradient over time for the compression pressure values C, indicated as dC/dt, indicating the pressure gradient over time of the ventilation pressure curve. For the different compression pressure values, this can be indicated as dC₁/dt and dC₂/dt respectively.
A spatial pressure gradient, indicated as dP/ds, indicating the difference in pressure as function of position, e.g. the spatial pressure gradient between P₁ and P₂.

In a third step 630a, 630b, 630c, a clinical parameter is determined based on the processed tracheal pressure values. Different clinical parameters can be determined as illustrated by steps 630a, 630b and 630c.

In a first example in step 630a it is evaluated whether the pressure gradient over time of the ventilation pressure curve surpasses a given threshold, indicated as Threshold 1. Such a threshold may be a value suitable for detecting the start of insufflation. The derived clinical parameter thus is whether or not the gradient over time of the ventilation pressure surpasses a given threshold. Depending on the fulfilment of the condition a diagnosis of insufflation may be made through judgment of relevantly trained people, as indicated in step 640a. For deriving further information, in step 650a, the ventilation parameters of the last ventilation may be determined, such as for example the area under the ventilation curve of ventilation pressure S₁, indicated as AUCV₁ the area under the ventilation curve of the ventilation pressure S₂, indicated as AUCV₂, the area under the ventilation curve for a negative ventilation pressure S₁ reflecting the duration and amplitude of negative detection for detection of gasping and spontaneous breathing, indicated as nAUCV₁, the positive end-expiratory pressure of the ventilatory curve for ventilation pressure S₁ and S₂, indicated as PEEPV₁ and PEEPV₂ respectively, the minimal tracheal pressures for P₁ and P₂ being the lowest detected pressure within the ventilation cycle which can be used for detection of gasping, the maximal spatial pressure gradient dP/ds, whereby dP is given by the difference in tracheal pressure P₁-P₂, the minimal spatial difference in tracheal pressure, i.e. the minimum dP, the moment of insufflation, the ventilation duration, the ventilation rate, etc. dP/ds thereby relates to the flow (e.g. in ml/sec) and thus can be used to determine the volumes of displaced air, i.e. the breathing volume.

In a second example in step 630b, it is evaluated whether the pressure gradient over time is below a given threshold, indicated as Threshold 2. Such a threshold may be a value suitable for detection of expiration. The derived clinical parameter thus is whether or not the gradient over time of the ventilation pressure is below the given threshold 2. Depending on the fulfilment of the condition a diagnosis of expiration may be made through judgment of relevantly trained people, as indicated in step 640b. For deriving further information, in step 650b, the ventilation parameters of the actual ventilation may be determined, such as for example the peak pressure of the ventilation pressure S₁ and S₂ which is the highest detected pressure within the ventilation cycle, the maximal pressure gradient over time for the ventilation pressure, which may be used for detection of oesophageal intubation, the minimal pressure gradient over time for the ventilation pressure, the duration of the insufflation, which may be used for evaluation of the quality of ventilation, etc.

In a third example in step 630c, it is evaluated whether the pressure gradient over time for the compression pressure surpasses a given threshold value, indicated as Threshold 3. Such a threshold may be a value suitable for detection of compression. Furthermore it is evaluated if, combined with the previous condition, the condition is fulfilled that the endotracheal pressure closest to the lungs P₁ is larger than the endotracheal pressure further away from the lungs P₂. The derived clinical parameter thus is whether or not the pressure gradient over time for the compression pressure is larger than a predetermined value and that P₁ is larger than P₂. Depending on the fulfilment of these conditions, a diagnosis of compression may be made through judgment of relevantly trained people, as indicated in step 640c. For deriving further information in step 650c, the compression parameters of the last compression also may be determined, such as for example the area under the compression curve of compression pressure C₁, indicated as AUCC₁ the area under the compression curve of the compression pressure C₂, indicated as AUCC₂, the maximal compression pressure C₁, the maximal compressive pressure gradient dC₁/dt for the compressive pressure values based on the endotracheal pressure values closest to the lungs, the moment of compression, the compression duration, the compression rate, etc.

In case compression is detected, the steps 650a and 650b may be performed using the ventilation pressure steps, whereas in other cases, the endotracheal pressure values may be used.

In step 660, the required results are outputted. In order to prevent a too large amount of information to be provided to the user, only the most relevant information may be provided to the user. Outputting also may be already partially performed after step 640a, 640b, 640c. One possible order of indication may be outputting information regarding oesophageal intubation, which is a function of the ventilation pressure gradients, the ventilation pressure values and the spatial gradient of the endotracheal pressure, then regarding the ventilation rate, then regarding respiration and/or gasping, which is a function of the minimal ventilation pressure, the minimal ventilation pressure gradient, the negative area under the ventilation curve and the difference between the endotracheal pressures, then regarding positive end expiratory pressure, then regarding the insufflation duration and the area under the curve per time, then regarding the compression rate and then regarding the pressure gradient during compression. The amount of info displayed may be selectable. The algorithm illustrates different aspects that may be implemented in software or hardware in systems of the present invention.

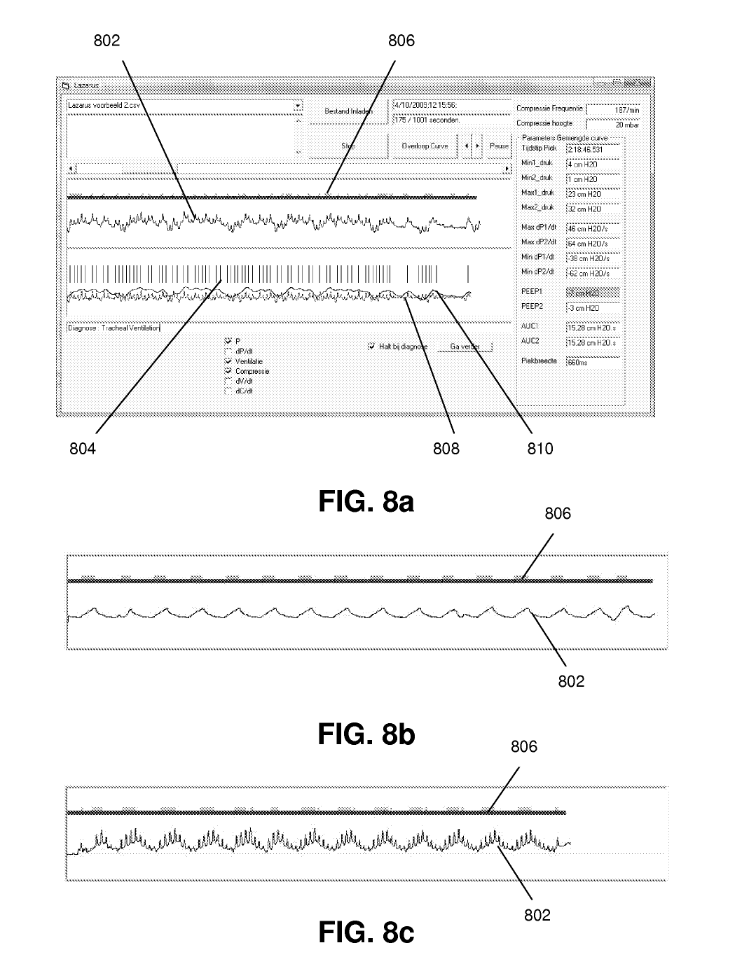

FIG. 8a, FIG. 8b and FIG. 8c illustrate an output window of software according to an embodiment of the present invention. In FIG. 8a, a recorded waveform 802 of CPR-pressure measurements is analysed. In the example shown, all relevant parameters are calculated in real time to determine physiological parameters. The thoracic compressions (stripes 804 in lower field) and insufflations (indicators 806 in upper field) are detected, the recorded waveform 802 is decomposed in a compression related pressure curve 808 and a ventilation related pressure curve 810. Analysis of the different parameters allows determination of the relevant physiological parameters. The system or associated software is adapted for informing the user if some of the parameters (see block diagram) are too different from the ideal values.
If multiple parameters are aberrant, a prioritizing algorithm is used to determine the most urgent and an alarm is given accordingly as was also discussed with reference to FIG. 7. FIG. 8b and FIG. 8c illustrate the insufflations (indicators 806) for both a mechanical ventilation without CPR and mechanical ventilation with CPR, as derived from the corresponding pressure curves 802.

It is to be understood that although preferred embodiments, specific constructions and configurations, as well as materials, have been discussed herein for devices according to the present invention, various changes or modifications in form and detail may be made without departing from the scope of this invention as defined by the appended claims.

1-19. (canceled)

20. A system for analysing resuscitation, the system comprising
an input means for receiving a plurality of tracheal pressure values over time for tracheal pressure during resuscitation,
a tracheal pressure value processing component for processing the obtained tracheal pressure values, the tracheal pressure value processing component being a tracheal pressure gradient calculation component for determining at least one tracheal pressure gradient value based on said obtained tracheal pressure values by calculating a pressure gradient value based on said obtained tracheal pressure values, and
a clinical parameter determination means configured to determine in real time at least one clinical parameter representative of a quality of resuscitation based on said processed tracheal pressure values.

21. A system according to claim 20, wherein the tracheal pressure gradient calculation component is configured to determine a temporal gradient in tracheal pressure values.

22. A system according to claim 20, the system being configured to analyse resuscitation using an endotracheal intubation tube, wherein the clinical parameter determination means is configured to determine whether the intubation tube is positioned oesophageal or tracheal based on said at least one tracheal pressure gradient value.

23. A system according to claim 20, wherein the clinical parameter determination means is configured to determine whether the tracheal pressure gradient value is higher than a first predetermined value.

24. A system according to claim 20, wherein the clinical parameter determination means is configured to evaluate sequential values of the tracheal pressure gradient value.

25. A system according to claim 20, wherein the clinical parameter determination means is configured to determine whether spontaneous cardiac activity is present.

26. A system according to claim 25, wherein the clinical parameter determination means is configured to detect at least two subsequent events of
a tracheal temporal pressure gradient value higher than a first predetermined value,
followed by a tracheal temporal pressure gradient value with absolute value lower than a second predetermined value,
followed by a high negative tracheal temporal pressure gradient value having an absolute value higher than a third predetermined value.

27. A system according to claim 20, the system being arranged to analyse resuscitation using an endotracheal intubation tube, wherein the tracheal pressure gradient calculation component is configured to determine a spatial gradient in tracheal pressure values based on tracheal pressure values obtained at different positions in an endotracheal intubation tube.

28. A system according to claim 20, wherein the clinical parameter determination means furthermore is configured to determine whether a maximal ventilatory pressure is below a fourth predetermined value.

29. A system according to claim 20, wherein the clinical parameter determination means furthermore is configured to determine a true compression.

30. A system according to claim 20, wherein the clinical parameter determination means is configured to determine whether a temporal pressure gradient value is above a fifth predetermined value, followed by a negative temporal pressure gradient value having an absolute value above a sixth predetermined value and wherein the highest pressure value is above a seventh predetermined value.

31. A system according to claim 20, wherein the system is configured to receive pressure values sensed within an endotracheal intubation tube.

32. A system according to claim 31, wherein the endotracheal intubation tube comprises a pressure sensor catheter having a catheter tube filled with air.

33. A system according to claim 20, wherein the system is part of a monitor, a ventilator or a defibrillator.

34. A method for analysing resuscitation, the method comprising
receiving a plurality of pressure values over time,
processing said obtained tracheal pressure values, and
determining in real time at least one clinical parameter representative of a quality of resuscitation based on said processed tracheal pressure values.

35. A method according to claim 34, wherein the method furthermore comprises assessing the resuscitation based on at least one clinical parameter and, if inappropriate, adapting the resuscitation.

36. A computer program product for, when executed on a computer, performing a method of analysing resuscitation, the method comprising
receiving a plurality of pressure values over time,
processing said obtained tracheal pressure values, and
determining in real time at least one clinical parameter representative of a quality of resuscitation based on said processed tracheal pressure values.

37. A computer program product according to claim 36, stored on a machine readable data storage device.

38. A computer program product according to claim 36, transmitted over a local or wide area telecommunications network.
